(19) United States
(12) Patent Application Publication
(10) Pub. No.: US 2026/0256181 A1
(43) Pub. Date: Sep. 3, 2026

(54) METHOD FOR PRODUCING A NUTRACEUTICAL DELIVERY SYSTEM

(71) Applicant: Exentis Knowledge GmbH, Stetten Ag (CH)

(72) Inventors: David L. Deck, Stetten Ag (CH); Ralf P. Brammer, Stetten Ag (CH)

(21) Appl. No.: 19/446,590

(22) Filed: Jan. 12, 2026

Related U.S. Application Data
(63) Continuation of application No. 16/958,922, filed on Dec. 19, 2018, now abandoned.

(30) Foreign Application Priority Data
Dec. 29, 2017 (DE) 10 2017 131 429.6

(51) Int. Cl. A23P 20/20; A23L 33/105; A23L 33/15; A23L 33/16; A23L 33/175; A23P 20/25; B33Y 10/00; B41F 15/40

(52) U.S. Cl. CPC A23P 20/20; A23L 33/105; A23L 33/15; A23L 33/16; A23L 33/175; B33Y 10/00; B41F 15/40; A23P 2020/253; A23V 2002/00

(57) ABSTRACT

The present invention refers to a method for producing a nutraceutical delivery system. The method comprises the steps of screen-printing a base paste, and curing the base paste. The method furthermore comprises the steps of screen-printing a first paste being separate to the base paste, and curing said first paste.

The present invention relates to a method for producing a nutraceutical delivery system. Preferably the nutraceutical delivery system produced by the method ac-cording to the invention is configured for a controlled, further preferred systemic administration of one or more active nutraceutical ingredients to a body, preferably a human body. The invention furthermore relates to a system for producing a nutraceutical delivery system.

A nutraceutical is commonly used to provide additional health benefit. An active nutraceutical ingredient may be the part of any nutraceutical that produces its effects, in particular any desired health effect. Some nutraceutical may have multiple active nutraceutical ingredients to provide different health benefits or act in different ways. Therefore, one or more active nutraceutical ingredients may be delivered by a nutraceutical. Even a higher number of nutraceutical ingredients may be delivered by a nutraceutical.

The delivery of nutraceuticals may refer to the transportation of a nutraceutical compound and/or ingredient into the body of a consumer as needed to safely achieve its desired effect, in particular its desired health effect. The delivery or administration of a nutraceutical into the body of a consumer, in particular a human, may be performed in various ways. Possible administration routes may include, for example, the oral route. In case delivery or administration into a body of a consumer is performed via the oral route, the respective nutraceutical is provided through the mouth of the consumer, in order to enter via the oral mucosa or pass on into the gastrointestinal tract to reach the blood compartment via the gastric or intestinal mucosa.

Nutraceuticals can be provided in different dosage forms. The dosage forms may comprise, for example, pills, tablets, capsules, solutions, dispersions and/or emulsions.

A tablet may be a nutraceutical dosage form. A tablet may be a solid unit dosage form of a nutraceutical with an active nutraceutical ingredient, with or without suitable excipients. Tablets may commonly be produced by molding or by compression. The manufacturing of tablets commonly requires that the appropriate amount of active nutraceutical ingredient(s) is provided in each tablet. This may be essential in view of safety as well as effectiveness of the respective tablet. All ingredients of a tablet should are therefore well mixed. Thereby, a homogeneous mixture of the ingredients may be obtained. Subsequently, a particular amount of the mixture may be compressed in order to obtain a tablet. Accordingly, the active nutraceutical ingredient is typically homogeneously distributed within and/or throughout the tablet, or parts of it.

With application of a tablet, for example upon oral administration, the tablet may dissolve. Thereby the active nutraceutical ingredient may be released. It may then pass the intestinal mucous membrane to reach the blood compartment and finally the tissue of action. With commonly produced nutraceutical delivery systems, for example with commonly produced tablets, the concentration of the active nutraceutical ingredient within the blood compartment may typically be above the efficacy threshold of the given active nutraceutical ingredient for a certain period of time. During this period of time, the release of the active nutraceutical ingredient out of the nutraceutical delivery system into the gastrointestinal tract may typically be much higher than actually required. Any excess amount of the active nutraceutical ingredient may not pass the membrane in sufficient amounts and be picked up by the body. Therefore, such excess amounts of the active nutraceutical ingredient may be excreted from the respective body. It is furthermore possible that excess amounts of the active nutraceutical ingredient reach the blood compartment and/or the tissue and cause possibly harmful effects.

In particular, release of active nutraceutical ingredients out of commonly designed and/or manufactured tablets is mainly driven by the size of the disintegrating tablet, in particular, the surface that is exposed to the surrounding fluid. As such it may be predefined by the form and size of the tablet and fixed, with, for example, a high release at the beginning and lowering over time. Blood and/or tissue concentrations of the active nutraceutical ingredient may thereby well exceed the respective efficacy threshold, in order to obtain a desired period of concentration above said threshold.

Such a release profile may particularly be disadvantageous for active nutraceutical ingredients with a narrow effective window. That is, in case of little differences between effective and harmful or excess doses, release profiles of common tablets may be particularly disadvantageous.

One problem underlying the present invention is to provide a method for producing a more efficient nutraceutical delivery system, which allows a controlled administration of one or more active nutraceutical ingredients to a body and/or with an application-tailored and/or active nutraceutical ingredient specific release profile.

A further object of the present invention is to provide a method for producing a nutraceutical delivery system which enables a controlled administration of several active nutraceutical ingredients to a body. A particular object is the controlled administration such that the active nutraceutical ingredients are released relative to each other in a defined manner, preferably with desired and/or adjusted active nutraceutical ingredient-specific release profiles.

A more general object of the invention may be considered as to provide an improved technique for producing a nutraceutical delivery system, which enables to produce advanced nutraceutical delivery systems with high quality and in great quantities. Any advanced nutraceutical delivery system may optimize kinetics and dynamics of the intake of the respective active nutraceutical ingredient.

These and other objects, which may become apparent from the following description, are solved by a method for producing a nutraceutical delivery system according to claim 1. A nutraceutical delivery system is subject to claim 33. Systems for producing a nutraceutical delivery system are subject to claims 34 and 35.

The present invention refers to a method for producing a nutraceutical delivery system. Said nutraceutical delivery system may enable the transporting of an active nutraceutical ingredient into the body of a consumer, preferably a body of a human, as needed to safely achieve its desired effect and/or a number of desired effects.

The nutraceutical delivery system according to the present invention may include an active nutraceutical ingredient, or several active nutraceutical ingredients, or other ingredients. The nutraceutical delivery system may be a bioerodible nutraceutical delivery system. Thus, the nutraceutical delivery system may erode upon application thereof to a body of a consumer. Accordingly, the nutraceutical delivery system may dissolve upon application, for example, in the mouth of the consumer.

The nutraceutical delivery system produced in accordance with the present invention may particularly be suited for a controlled administration of one or more active nutraceutical ingredients to a body. The body may be the body of a consumer, such as, for example, a human. It is also possible that the body is the body of an animal.

The nutraceutical delivery system produced in accordance with the present invention may be used for oral administration of one or more active nutraceutical ingredients to a body. The nutraceutical delivery system may dissolve in the mouth of the consumer. Therefore, with the nutraceutical delivery system produced in accordance with the present invention, an active nutraceutical ingredient can be administered in a controlled manner. The manner of controlled administration may depend on the particular application case and/or the health effects to be achieved.

In accordance with the present invention, the method for producing a nutraceutical delivery system comprises the step of screen-printing a base paste. The base paste may, for example, include and/or be composed of water, polyvinylpyrrolidone, citric acid, hypromellose, stearate, silic acid, glycerol, hydroxypropyl cellulose, hydroxypropyl methylcellulose, starch, cellulosecrosscaramelose, glycol, crystalline gelatin, collagen, hydroxyapatite, hydrocarbonate, lactide, lactic acid, silica, polaxamers, xylitol, erythritol, ethanol, isopropanol, triacetin, aspartame, sodium bicarbonate, and/or acetone. The viscosity of the base paste may, for example, be in the range of 1×10^{-2 to} 1×10¹⁴ mPa·s, preferably in the range of 1×10^{-1 to} 1·10⁸ mPa·s. The viscosity of the base paste may, more preferably, be in the range of 1×10⁰ to 1×10⁷ mPa·s, more preferably in the range of 1×10¹ to 1·10⁶ mPa·s. For screen-printing the base paste, a respective printing mesh may, for example, be used. Such a printing mesh allows for providing the base paste in accordance with a desired printing profile. Accordingly only certain areas of the resulting nutraceutical delivery system may be formed of the base paste.

The method according to the present invention furthermore comprises the step of curing the base paste. The base paste may particularly be cured such that it hard-ens. The curing temperatures and curing times may, for example, depend on the composition of the base paste. The base paste may, for instance, be cured at a temperature of 30° C. to 180° C., preferably 35° C. to 150° C., more preferably 40° C. to 110° C., more preferably 45° C. to 90° C., more preferably 50° C. to 70° C. It is also possible to apply curing times of 10 seconds to 1 hour, preferably 30 seconds to 30 minutes, more preferably 1 minute to 10 minutes.

The method according to the present invention furthermore comprises the step of screen-printing a first paste, said first paste being separate to the—preferably cured—base paste. Accordingly, the first paste may be provided separate from the base paste. The first paste may thereby be arranged separate from the base paste, that is, preferably without any overlap. This due to the fact that the first paste may be screen-printed such that it is arranged at locations where the base paste was not screen-printed. In other words, the first paste may be screen-printed such that it is arranged at locations free of the base paste.

According to the present invention, the component(s) of the first paste are not mixed with the component(s) of the base paste in a classical manner to form a homogeneous mixture. To the contrary, the first paste is provided separate to the base paste. Within the resulting nutraceutical delivery system, the base paste may accordingly be distinguished from the first paste. The first paste may, for example, include and/or be composed of water, polyvinylpyrrolidone, citric acid, hypromellose, stearate, silic acid, glycerol, hydroxypropyl cellulose, hydroxypropyl methylcellulose, starch, cellulosecrosscaramelose, glycol, crystalline gelatin, collagen, hydroxyapatite, hydrocarbonate, lactide, lactic acid, silica, polaxamers, xylitol, erythritol, ethanol, isopropanol, triacetin, aspartame, sodium bicarbonate, and/or acetone. The viscosity of the base paste may be in the range of 1×10^{-2 to} 1·10¹⁴ mPa·s, preferably in the range of 1×10^{-1 to} 1·10⁸ mPa·s, more preferably in the range of 1·10⁰-1·10⁷ mPa·s, more preferably in the range of 1·10¹ to 1·10⁶ mPa·s. A printing mesh may be used for screen-printing the first paste. The printing mesh may allow for providing the first paste in accordance with a desired printing profile, so that for example only certain areas of the resulting nutraceutical delivery system are formed of the first paste.

The method according to the present invention furthermore comprises the step of curing the first paste. The curing temperatures and curing times may, for example depend on the composition of the first paste. The first paste may, for instance, be cured at a temperature of 30° C. to 180° C., preferably 35° C. to 150° C., more preferably 40° C. to 110° C., more preferably 45° C. to 90° C., more preferably 50° C. to 70° C. Furthermore, curing times of preferably 10 seconds to 1 hour, more preferably 30 seconds to 30 minutes, more preferably 1 minute to 10 minutes may be applied. The first paste may, for instance, be cured together with the base paste. The screen-printing and curing of the first paste may, as an alternative, be performed after curing the screen-printed base paste.

In accordance with the present invention, the first paste comprises an effective amount of a first active nutraceutical ingredient. The first paste may, therefore, comprise the active nutraceutical ingredient, said ingredient to be delivered or administered by means of the resulting nutraceutical delivery system. Furthermore, the first active nutraceutical ingredient may be homogeneously distributed within and/or throughout the first paste. It may be comprehended that the first paste may comprise several active nutraceutical ingredients. Several active nutraceutical ingredients may again be homogeneously distributed within and/or throughout the first paste. It is also possible that the base paste comprises an active nutraceutical ingredient.

The method according to the present invention enables producing an enhanced nutraceutical delivery system. The base paste and the first paste may in accordance with the present invention be provided in the nutraceutical delivery system in a manner such that it is possible to obtain a particularly desired and/or adjusted release of the first active nutraceutical ingredient. Controlling the arrangement of the first paste in relation to the base paste during the respective screen-printing steps, that is, by choosing suitable printing profiles, it may be controlled at what time and at which rate the first active nutraceutical ingredient is released from the nutraceutical delivery system. Therewith a nutraceutical delivery system may be produced, which enables an optimal active nutraceutical ingredient release for a controlled administration of a respective active nutraceutical ingredient to a body.

The utilization of the screen-printing technique allows for the mass production of the nutraceutical delivery system, while at the same time ensuring a high precision. As an example, nano-sized geometries of the first paste, and thus of the first active nutraceutical ingredient, may be printed. The arrangement of the first active nutraceutical ingredient within and/or throughout the resulting nutraceutical delivery system may be controlled at high precision.

The screen-printing technique also allows providing the first paste in a manner such that is forms a particularly preferred geometrical shape in the cured state and/or in the resulting nutraceutical delivery system. The utilization of the screen-printing technique furthermore allows the parallel production of several nutraceutical delivery systems. As an example, in the course of screen-printing the base paste, numerous nutraceutical delivery systems may be produced at the same time by using a respective printer with a mesh allowing for printing the base paste to form an array of 100×100 tablets, for example. Also the first paste may be printed to eventually form the array of 100×100 tablets simultaneously. The array of 100×100 tablets may likewise be cured simultaneously.

The resolution of the screen-printing pattern may, for instance, depend on the composition of the pastes. A resolution, for example, in the range of 10 dpi to 10000 dpi, preferably 100 dpi to 5000 dpi, more preferably 200 dpi to 2000 dpi, more preferably 500 dpi to 1000 dpi may be provided for. The first active nutraceutical ingredient may therefore eventually be arranged in the nutraceutical delivery system in a refined manner. Thus, two-or three-dimensional structures formed of the base paste and the first paste in the nutraceutical delivery system may feature a resolution, for example, in the range of 10 dpi to 10000 dpi, preferably 100 dpi to 5000 dpi, more preferably 200 dpi to 2000 dpi, more preferably 500 dpi to 1000 dpi.

According to a preferred embodiment of the present invention, the nutraceutical delivery system may be produced layer-by-layer. By producing the nutraceutical delivery system in a layer-by-layer manner, one layer may be formed on top of another layer. Thereby, the nutraceutical delivery system may be build up. A first layer of the nutraceutical delivery system may, for example, be produced by screen-printing and curing the base paste and the first paste. Subsequently, a further layer may be produced on top of the first layer. This sequence may be repeated several times.

The nutraceutical delivery system may, according to a further preferred embodiment, be produced using a movable platform. Said platform may, for example, be provided underneath a printing screen. After the completion of each layer, the movable platform may be lowered vertically by a respective step size. Subsequently the next layer may be produced on top thereof. It may be comprehended that the arrangement of the first paste, which is possibly cured, relative to the base paste, which is possibly cured, may differ in adjacent layers.

According to a further preferred embodiment, the pastes are screen-printed in a manner such that a resulting planar layer of the nutraceutical delivery system comprises both the cured base paste and the cured first paste. A planar layer of the resulting nutraceutical delivery system may therefore comprise the cured base paste. Said planar layer may also comprise the cured first paste, said first paste being separate to the base paste. Accordingly, both pastes—the pasted being in a cured state—may be differentiated from another. This is due to the fact that no homogeneous mixture is provided.

According to a further preferred embodiment, the planar layer of the nutraceutical delivery system may be produced by screen-printing and curing the base paste to partially form the planar layer, and by screen-printing and curing the first paste, said first paste being separate to the base paste, to partially form the planar layer. By producing the planar layer, the pastes may not be screen-printed in an overlapping manner. Accordingly, by screen-printing and curing the base paste, a part or portion of the resulting planar layer may be formed. A further part or portion of the resulting planar layer, preferably the remaining part or portion of the resulting planar layer, may subsequently be formed by screen-printing and curing the first paste. As an example, the resulting planar layer may comprise areas or portions where only the base paste is arranged, for instance at outer regions or portions of the layer. The resulting planar layer may furthermore comprise areas or portions where only the first paste is arranged, for instance, at inner regions or portions of the layer. The pastes therefore do not necessarily form continuous areas. Quite contrary, the pastes may form separate areas, such as “islands”.

According to a further preferred embodiment, after finishing the production of the planar layer, a further planar layer may be produced on top of the finished planar layer. A different arrangement or printing-profile may be chosen in this manner. Accordingly, a desired three-dimensional arrangement of the first paste relative to the base paste may be obtained. As a consequence, a desired three-dimensional distribution of the first active nutraceutical ingredient throughout the resulting nutraceutical delivery system may eventually be obtained.

According to a yet further preferred embodiment of the present invention, the base paste may be screen-printed using a screen-printer, and the first paste may be screen-printed using a separate screen-printer. I In a respective production line, several screen-printers may therefore be arranged. The screen-printers may each be configured for printing a single paste, for instance, the base paste or the first paste. The production line can be modified to produce different nutraceutical delivery system designs in accordance with the present invention, by inserting or removing individual printers into or out of the production line. Accordingly, a high flexibility may be achieved by such modular setup.

According to a further preferred embodiment of the present invention, the base paste and the first paste may be cured with a shared or the same curing device. Therefore, only one curing device may be required for the production line, for producing the nutraceutical delivery system in accordance with the present invention. Even though several individual screen-printers may be used for producing a nutraceutical delivery system, the built may be transferred to the shared curing device, in order to cure the respective paste(s). Cost savings may therewith be achieved.

According to a yet further preferred embodiment of the present invention, the base paste—the base paste being in a cured state—and the first paste—the first paste being in a cured state—may be soluble in body fluids. Body fluids within the meaning of the present invention may, for instance, include blood, and/or body tissue fluids. Body fluids encountered may vary according to the administration route. With oral intake of the nutraceutical delivery system, the composition of the outer layer may determine whether dissolution of the nutraceutical delivery system will begin in the mouth, thus with dissolution beginning in saliva, or later along the journey of the device through the gastrointestinal tract, in particular the stomach, thus in acidic milieu, the ileum, the jejunum or other places. It may be comprehended that the dissolution characteristics of the pastes, that is, the cured pastes, and thus of the resulting nutraceutical delivery system may be chosen or adjusted in a manner such that a suitable release of the active nutraceutical ingredient may be obtained depending on the respective application. A rather instant or a rather slow dissolution may be chosen or adjusted accordingly.

The first paste, that is the cured first paste, and the base paste, that is the cured base paste, may dissolve in a similar manner. Both the base paste and the first paste may preferably dissolve in the same body fluid. By screen-printing the first paste and the base paste separate to one another, and in view of the dissolution characteristics thereof, it may be well controlled at what time and at which rate the first active nutraceutical ingredient is released from the resulting nutraceutical delivery system to the respective body or body fluid. The release of the active nutraceutical ingredient may preferably be determined only by the dissolution characteristics of the cured pastes, and/or the form and/or the shape of the resulting nutraceutical delivery system. Further release agents, such as, for example, osmotic agents for releasing the active nutraceutical ingredient, may not be required.

According to a further preferred embodiment of the present invention, the pastes may be screen-printed such that in the resulting nutraceutical delivery system, the first paste, that is, the cured first paste, is inhomogeneously arranged in the base paste, that is, the cured base paste. The base paste and the first paste may therefore not be provided as a homogeneous mixture in the resulting nutraceutical delivery system. To the contrary the base paste and the first paste may be provided separately from another, preferably in a manner, according to which the first paste is inhomogeneously arranged in the base paste. The first paste may be provided inhomogeneously or discontinuously along one, two or more preferably three spatial or orthogonal directions within or throughout the base paste. Thereby, the first paste may be arranged in the resulting nutraceutical delivery system in such a controlled, adjusted and/or desired manner, so that no homogeneous distribution of the first paste, and thus also of the first active nutraceutical ingredient, is present within or throughout the resulting nutraceutical delivery system. To the contrary, the inhomogeneity may be specifically constituted by the particular arrangement of the pastes. Since the base paste and the first paste are provided as separate pastes, particularly by separately screen-printing the base paste and the first paste in a preferably non-overlapping manner, the first paste can be arranged inhomogeneously within and/or throughout a matrix formed of the base paste. The amount of the first paste arranged within and/or throughout the base paste may, for instance, increase gradually along a particular direction throughout the resulting nutraceutical delivery system and/or a portion of the resulting nutraceutical delivery system.

According to a yet further embodiment of the present invention, the pastes may be screen-printed such that in the resulting nutraceutical delivery system, the base paste, in particular the cured base paste, may be provided or considered as a three-dimensional body. Furthermore, the first paste, in particular, the cured first paste, may be inhomogeneously arranged within and/or throughout the base paste. The main body of the resulting nutraceutical delivery system may accordingly be formed of the base paste and one or more particular parts of the nutraceutical delivery system, which may, for instance, be only of marginal size, may be formed of the first paste.

According to a yet further preferred embodiment of the present invention, the first component may inhomogeneously be arranged in a three-dimensional manner. Thus, the first component may be inhomogeneously arranged in the base component, and at the same time the inhomogeneous arrangement may be provided three-dimensionally, that is, throughout a three-dimensional extent of the of the base component.

The base paste and the first paste may furthermore be arranged on a virtual two- or three-dimensional grid. Each pixel of the grid may be occupied by the base paste or by the first paste. The first paste may therefore be preferably arranged in an inhomogeneous manner within and/or throughout the base paste. Accordingly, the first paste may be inhomogeneously arranged within and/or throughout the resulting nutraceutical delivery system itself. Such a pixel may have a size or volume in the range of 1 μm³ to 1 cm³, preferably in the range of 10 μm³ to 100 mm³, more preferably in the range of 100 μm³ to 10 mm³, and even more preferably of about 1 mm³.

According to the present invention, the principle of a homogeneous distribution of an active nutraceutical ingredient throughout the nutraceutical delivery system may be suspended. It is therefore possible to provide a particular arrangement of the active nutraceutical ingredient within and/or throughout the resulting nutraceutical delivery system, in order to obtain a nutraceutical delivery system with a specifically adjusted or customized release profile of the active nutraceutical ingredient. The paste with the active nutraceutical ingredient may, for instance, be arranged in a manner such that a steady release of the active nutraceutical ingredient is obtained. The release may preferably result in a blood-tissue concentration above or slightly above the efficacy threshold of the active nutraceutical ingredient. In comparison to the commonly produced nutraceutical delivery systems, which have a homogeneous distribution of the active nutraceutical ingredient, a preferred nutraceutical delivery system produced according to the present invention requires an amount of active nutraceutical ingredient being effectively less. In addition to this, the same physiological results and/or desired health-effects are maintained with lower side-effects.

According to a yet further preferred embodiment of the present invention, the inhomogeneous distribution of an active nutraceutical ingredient in the nutraceutical delivery system produced according to the present invention may be utilized in a standardized manner. A particular arrangement and/or adjustment may be chosen or set, as required for the respective application. Such a concept allows for producing nutraceutical delivery systems with advantageous release profiles or release characteristics as described herein. The standardization, definition and/or specification of the arrangement of the pastes and therefore also, the standardization, definition or specification of the inhomogeneity of the active nutraceutical ingredient enables the production of such nutraceutical delivery systems uniformly in high quantity, and at the same time also in a mass production.

The release profile and/or release characteristic of the active nutraceutical ingredient may be, for example, be configured and/or adjusted such that release of the active nutraceutical ingredient at a constant rate over a prolonged period of time is be provided. In other scenarios, a particularly slow release of an active nutraceutical ingredient to a body, with a release rate slightly above the efficacy threshold of the active nutraceutical ingredient, may be provided. The rate of release may in this case be approximately independent of time. According to a further preferred scenario, the release profile and/or characteristic may be configured for release of the active nutraceutical ingredient at particular intervals, for example, intermittently over time. According to yet a further preferred scenario, the release of several active nutraceutical ingredients one after the other, or simultaneously at individual release rates, may be provided, particularly with active nutraceutical ingredient-specific release profiles and/or characteristics.

It may be comprehended that by screen-printing and curing the base paste, processes such as cross-linking within the base paste may take place. Eventually the base paste itself may be altered in this manner. It may further be comprehended that the resulting structure of the cured base paste may still be considered to be essentially formed of the respective base paste, even though its viscosity may have changed significantly. Thus, when reference is made to the base paste in the resulting nutraceutical delivery system, it may be comprehended that this base paste may be the respective cured base paste. This also applies to other pastes with the system.

According to a yet a preferred embodiment of the present invention, the pastes may be screen-printed in manner such that in the resulting nutraceutical delivery system the concentration of the first active nutraceutical ingredient varies within and/or throughout the nutraceutical delivery system. In particular, the concentration may vary throughout the body defined by the base paste. Printing profiles may, for example, be chosen such as to allow for screen-printing the first paste at central parts of the nutraceutical delivery system only. Accordingly, particular regions or portions of the resulting nutraceutical delivery system may be identified having a comparably high concentration of the first active nutraceutical ingredient. On the other hand, particular regions or portions may be identified having a comparably low or even no concentration of the first active nutraceutical ingredient. While taking into consideration the particular form and/or shape of the nutraceutical delivery system, as well as the dissolution characteristics of the base paste and first paste, it may be precisely controlled when and how the active nutraceutical ingredient is released eventually.

According to a yet further preferred embodiment of the present invention, the pastes may be screen-printed in a manner such that in the resulting nutraceutical delivery system the concentration of the first active nutraceutical ingredient is relatively high or highest at a center, at an edge or at an intermediate region of the nutraceutical delivery system. As an example, if the resulting nutraceutical delivery system is provided in the form of a tablet, the first paste may be arranged or screen-printed in manner such that a peak concentration of the first active nutraceutical ingredient is provided at a center or a central part of the tablet. With administration of such a tablet and subsequent dissolution of the base paste and the first paste, the release of the first active nutraceutical ingredient may increase over time. It is also possible that the release of the first active nutraceutical ingredient may remain approximately constant over time. This may depend on the shape of the nutraceutical delivery system. In this manner, a specific and/or desired release of the active nutraceutical ingredient may be obtained.

According to a yet further preferred embodiment of the present invention, the pastes may be screen-printed in a manner such that in the resulting nutraceutical delivery system a gradient of the concentration of the first active nutraceutical ingredient increases towards or increases away from a center or a center portion of the nutraceutical delivery system. The printing profiles may, for instance, be chosen in a manner such that the amount of screen-printed first paste increases towards the center or a center portion of the nutraceutical delivery system. In case the resulting nutraceutical delivery system is provided in form of a spherical tablet and in case the concentration increases towards the center of the tablet, the arrangement of the first paste and therefore of the first active nutraceutical ingredient may be provided in a manner such that the release rate is approximately constant with application of the nutraceutical delivery system. Adjusting the concentration profile of the active nutraceutical ingredient within and/or throughout the nutraceutical delivery system by adjusting the printing profile during the respective screen-printing step or steps, the release profile of the active nutraceutical ingredient may be well controlled.

According to a yet further preferred embodiment of the present invention, the pastes may be screen-printed in a manner such that in the resulting nutraceutical delivery system a concentration profile of the first active nutraceutical ingredient within and/or throughout the nutraceutical delivery system comprises a smooth transition to an area or portion of increased concentration. The printing profiles may, for example, be chosen in a manner such that the amount of screen-printed first paste increases gradually towards the center or center portion of the nutraceutical delivery system. Accordingly, the concentration profile may have a smooth transition between an area of low or even no concentration on the one hand, and an area of high or comparably high concentration on the other hand. A smooth transition within the meaning of the present invention may be defined by the absence of any abrupt or any discontinuous steps in the resulting concentration profile. The concentration profile may, within the meaning of the present invention, represent the profile of the concentration of the first active nutraceutical ingredient diagonally across the resulting nutraceutical delivery system. The concentration profile may, for example, represent the profile of the contration from one edge of the nutraceutical delivery system to its center or a center portion, or possibly extending through the entire nutraceutical delivery system. With smooth transitions, as mentioned above, it shall be possible to obtain a smooth onset of the release of the active nutraceutical ingredient with dissolution of the respective cured paste.

According to a yet further preferred embodiment of the present invention, the pastes may be screen-printed in a manner such that in the resulting nutraceutical delivery system a concentration profile of the first active nutraceutical ingredient within and/or throughout the nutraceutical delivery system comprises more than one area of comparably high or increased concentration. With such a resulting nutraceutical delivery system, several dosages of the active nutraceutical ingredient may thereby be administered over time. More preferably, as a result of to the respective printing profiles during screen-printing, the deposition of the first active nutraceutical ingredient within and/or throughout the nutraceutical delivery device along the dissolution direction, that is, from the periphery to the center, may be discontinuous and/or repetitive in an onion skin type manner. Within each such shell of the nutraceutical delivery system, the first paste may be provided inhomogeneously. Accordingly, a release of the first active nutraceutical ingredient may preferably not start in an abrupt manner. Instead the release can be set in manner such it starts and/or ends gradually. The release of the active nutraceutical ingredient may thereby be provided in distinct waves. Thus, intervals with high release of the first active nutraceutical ingredient may be followed by intervals with low or no release. The active nutraceutical ingredient may furthermore be administered in several phases over time. These phases—in particular also their onset—may be controlled by controlling the arrangement of the areas of high or increased concentration within and/or throughout the nutraceutical delivery system, particularly by choosing or suitably adjusting the respective printing profiles during screen-printing.

According to a yet further preferred embodiment of the present invention, the pastes may be screen-printed such that in the resulting nutraceutical delivery system the variation of the concentration of the first active nutraceutical ingredient within and/or throughout the system is at least 5%, preferably at least 10%, more preferably at least 15%, more preferably at least 20%, more preferably at least 25%, more preferably at least 30%, more preferably at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably approximately 100%.

According to a yet further preferred embodiment of the present invention, the pastes may be screen-printed in a manner such that in the resulting nutraceutical delivery system the variation of the concentration of the first active nutraceutical ingredient within and/or throughout the system is at most approximately 100%, preferably at most 95%, more preferably at most 90%, more preferably at most 85%, more preferably at most 80%, more preferably at most 75%, more preferably at most 70%, more preferably at most 65%, more preferably at most 60%, more preferably at most 55%, more preferably at most 50%, more preferably at most 45%, more preferably at most 40%, more preferably at most 35%, more preferably at most 30%, more preferably at most 25%, more preferably at most 20%, more preferably at most 15%, more preferably at most 10%, more preferably at most 5%. Accordingly, the variation of the concentration may be set in a controlled manner, particularly by providing a respective local arrangement of the first paste relative to the base paste by using the screen-printing technique, in order to eventually obtain a desired and/or specifically controlled administration of the first active nutraceutical ingredient. The variation of the concentration of the first active nutraceutical ingredient may, within the meaning of the present invention, be defined as the difference of the maximum concentration and the minimum concentration of the active nutraceutical ingredient within the nutraceutical delivery system. In the present context, the concentration may, for instance, be the mass-specific concentration. The respective sampling volume for measuring the concentration may be any suitable volume. A suitable may, for example, be of 1 μm³ . As an example, in case the highest concentration within a sampling volume in the nutraceutical delivery system is of about 80% on the one hand, and the lowest concentration in a sampling volume within the nutraceutical delivery system is of about 10%, the variation may thus be 70%. For instance, throughout the nutraceutical delivery system, the concentration of the first active nutraceutical ingredient may be at least 10%. At a central part of the nutraceutical delivery system, the concentration of the first active nutraceutical ingredient may increase to 80%.

According to a yet further preferred embodiment of the present invention, the pastes may be screen-printed in manner such that in the resulting nutraceutical delivery system the concentration profile of the first active nutraceutical ingredient is such that with application of the system, the first active nutraceutical ingredient is released from the system at a predetermined release profile. The release profile may more preferably comprise a section with a release at a constant rate. Due to the particular screen-printing profiles, the first paste may be arranged in manner within the base paste such that with application of the resulting nutraceutical delivery system, and with dissolution of the cured base paste and the cured first paste, a particular or specifically adjusted release profile of the active nutraceutical ingredient may be obtained. The release profile may comprise a constant release section according to a preferred embodiment.

According to a yet further preferred embodiment of the present invention, the pastes may be screen-printed in a manner such that in the resulting nutraceutical delivery system the first paste is arranged in the base paste such that with dissolution of the nutraceutical delivery system or the cured pastes respectively, the total amount of the first active nutraceutical ingredient at an outer surface of the nutraceutical delivery system remains approximately constant for a predetermined time. The predetermined time may preferably be in the range of 1 second up to 24 hours. Printing profiles may, for example, be chosen allowing for screen-printing the first paste in a manner such that the amount of the printed first paste increases towards the center or central parts of the nutraceutical delivery system only. It may be comprehended that depending on the respective application and the form of the nutraceutical delivery system, comparably longer or comparably shorter release periods may be applicable.

In case the nutraceutical delivery system is produced in form of a tablet, the active nutraceutical ingredient may be released during a period of up to 12 hours, for example. Preferably, the predetermined time of approximately constant release may be in the range of 5 seconds to 24 hours, more preferably 10 seconds to 12 hours, more preferably, 1 minute to 6 hours, more preferably 10 minutes to 1 hour. In the exemplary case of a spherical tablet, a gradient of the concentration of the first active nutraceutical ingredient may point inwards. Therefore the amount of active nutraceutical ingredient at the surface of the nutraceutical delivery system may remain constant at the time the nutraceutical delivery system is dissolving, thus, when the volume and surface of the system shrinks. The first paste may therefore be arranged in a manner such that eventually the concentration of the first active nutraceutical ingredient depends on the distance to the outer surface of the nutraceutical delivery system. A constant release of the first active nutraceutical ingredient may therefore be set by inhomogeneously arranging the first paste within the base paste with the screen-printing technique.

According to a yet further preferred embodiment, the pastes may be screen-printed in a manner such that in the resulting nutraceutical delivery system the concentration profile of the first active nutraceutical ingredient is such that with application of the system, the first active nutraceutical ingredient is released at two or more dosages. The release of the first active nutraceutical ingredient at one of the dosages starts preferably 1 second to 24 hours, more preferably 5 seconds to 12 hours, more preferably 10 seconds to 6 hours, more preferably 20 seconds to 2 hours, more preferably 1 minute to 1 hour, and even more preferably 10 minutes to 30 minutes before release of the first active nutraceutical ingredient at another one of the dosages.

Printing profiles may, for instance, be chosen in manner such that the first paste is provided at several, separated locations towards a center or central portion of the nutraceutical delivery system. If the nutraceutical delivery system is, for example, provided in form of a tablet, and with oral administration of the tablet, the first active nutraceutical ingredient may be released at a first dosage shortly after administration. Thus, the first dosage may be release before the first active nutraceutical ingredient is released at a second dosage at a later time. It is possible that the dosages are uniform or vary among each other. The duration of release of any active nutraceutical ingredient mentioned herein may be measured by means of dissolution tests. A dissolution test may, for example, be conducted according to USP-Guideline “General Chapter <711> Dissolution”.

According to a yet further preferred embodiment of the present invention, the pastes may be screen-printed in a manner such that in the resulting nutraceutical delivery system the base paste envelops and/or encloses the system and the first paste is not arranged at an outer face of the system. That is, the outer face of the system may be free of the first paste. In other words, the first paste with the first active nutraceutical ingredient may be provided in manner such that it cannot be accessed from the outside or outer surface of the system, at least prior to the application of the nutraceutical delivery system. Accordingly, the first active nutraceutical ingredient can be sealed and/or protected from the environment, therewith reducing the risk of contamination.

In case the nutraceutical delivery system is, for example, produced in form of a tablet, the dissolution of the first paste may be delayed with oral administration. This results from the fact that the base paste has to dissolve first or at least partially dissolve first. Accordingly, a delayed administration of the first active nutraceutical ingredient may be obtained, respectively. The nutraceutical delivery system may preferably be produced in a manner such that release of the first active nutraceutical ingredient starts 1 second to 24 hours, preferably 10 seconds to 12 hours, more preferably 30 seconds to 6 hours, more preferably 1 minute to 4 hours, more preferably 10 minutes to 2 hours, more preferably 30 minutes to 1 hour after application of the respective nutraceutical delivery system.

According to a yet further preferred embodiment of the present invention, the method may further comprise the steps of screen-printing a second paste being separate to the base paste and the first paste, and curing said second paste. The second paste may, for example, comprise an effective amount of a second active nutraceutical ingredient. The method therefore allows to produce a nutraceutical delivery system enabling a controlled administration of several active nutraceutical ingredients in particular applications. The active nutraceutical ingredients may interact after dissolution of the respective paste. The ingredients may thus provide a synergetic effect or different synergistic effects within the body of the consumer. The first and second active nutraceutical ingredient may preferably differ in form as well as in concentration. The ingredients may also have the same form and concentration. The cured second paste may preferably be soluble in body fluids as well. The respective provisions and explanations given with reference to the first paste and the base paste may similarly apply with reference to the second paste.

It may be comprehended that the provisions and explanations given herein with regard to the screen-printing and curing steps of the base paste and first paste, as well as the provisions and explanations regarding the first active nutraceutical ingredient may similarly apply analogously to the second paste and the second active nutraceutical ingredient. It may further be comprehended that the method may comprise further steps of screen-printing and curing further pastes with further active nutraceutical ingredients, for example a third paste comprising a third active nutraceutical ingredient, and/or a fourth paste comprising a fourth active nutraceutical ingredient. Further pastes with further active nutraceutical ingredient may be screen-printed and subsequently cured.

According to a yet further preferred embodiment, the pastes may be screen-printed in a manner such that a resulting planar layer of the nutraceutical delivery system comprises all of the cured base paste, the cured first paste and also the cured second paste. A planar layer of the resulting nutraceutical delivery system may therefore comprise the cured base paste, the cured first paste being separate to the base paste, and also the cured second paste being separate to the base paste and the first paste. Accordingly, all of the cured pastes may be differentiated from another. This is due to the fact that no homogeneous mixture is provided.

According to a yet further preferred embodiment of the present invention, the planar layer of the nutraceutical delivery system may be produced by screen-printing and curing the base paste in order to partially form the planar layer, screen-printing and curing the first paste being separate to the base paste in order to partially form the planar layer, and screen-printing and curing the second paste being separate to the base paste and the first paste in order to partially form the planar layer.

By producing the planar layer, the pastes are preferably not screen-printed in an overlapping manner. By screen-printing and curing the base paste, a part of the resulting planar layer may be formed respectively. A further part of the resulting planar layer may subsequently be formed by screen-printing and curing the first paste. A yet further part of the resulting planar layer, which is preferably the remaining part of the resulting planar layer, may subsequently be formed by screen-printing and curing the second paste. As an example, the resulting planar layer may comprise areas where only the base paste is arranged, areas where only the first paste is arranged, and areas wherein only the second paste is arranged. Areas where only the base paste is arranged may, for example, be outer regions of the layer. Areas where only the first paste is arranged may, for example, be inner regions of the layer. Areas wherein only the second paste is arranged may, for example, be intermediate regions of the layer. The pastes must not necessarily form continuous areas. Instead the pastes may form separate areas, such as “islands”.

According to a yet further preferred embodiment of the present invention, the pastes may be screen-printed in a manner such that in the resulting nutraceutical delivery system the cured second paste is inhomogeneously arranged in the cured base paste. Accordingly, the release of the first active nutraceutical ingredient and the second active nutraceutical ingredient from the nutraceutical delivery system may be controlled also relatively to each other. This may be possible by controlling the inhomogeneous arrangement of the respective first and second pastes in the base paste. The explanation above with regard to the inhomogeneous arrangement likewise applies here.

According to a yet further preferred embodiment of the present invention, the pastes may be screen-printed in a manner such that in the resulting nutraceutical delivery system a concentration profile of the first active nutraceutical ingredient within and/or throughout the nutraceutical delivery system is different than a concentration profile of the second active nutraceutical ingredient within and/or throughout the nutraceutical delivery system. Printing profiles may, for example, be chosen such that the amount of screen-printed first paste increases towards a center or central portion of the nutraceutical delivery system. In addition, the amount of screen-printed second paste may decrease towards the center or the central portion of the nutraceutical delivery system. The nutraceutical delivery system may therefore be designed and/or produced in a manner such that the first active nutraceutical ingredient and the second active nutraceutical ingredient are released to the respective body of the consumer at different dosages.

According to a yet further preferred embodiment of the present invention, the pastes may be screen-printed in a manner such they are eventually arranged in a discontinuous manner within and/or throughout the resulting nutraceutical delivery system. The discontinuous may be in a manner such that the first active ingredient may be released for a distinct period of time with the start of the dissolution of the nutraceutical delivery system. The start of dissolution typically occurs from its periphery. As in case of an onion skin type arrangement, the layer with the first paste may be adjacent to a further layer with either no active nutraceutical ingredient or the second active nutraceutical ingredient, as an example. The release of the active nutraceutical ingredients may be controlled by varying parameters, as for example, the thickness of the layers, their composition, and the distribution of the active nutraceutical ingredients within said layers.

According to a yet preferred embodiment, at least one layer of the nutraceutical delivery system may be resistant to dissolution in certain body fluids or certain body environments and/or may dissolute in certain body fluids or environments at in a delayed manner. It is also possible that at least one layer of the nutraceutical delivery system may be resistant to dissolution in certain body fluids or environments and may dissolute in other body fluids or environments. Thus, at least one layer may have dissolution resistant or dissolution delaying properties. A plurality of such layers may be provided. Such layers may, for example, be arranged on two sides of a possible third layer in a sandwich type configuration. In this case, the third layer may, for example, only be subject to dissolution from the narrow side or from its edges. The remaining sides or surfaces are protected from being contacted by body fluids or tissue due to the arrangement of the further layers, which do not dissolute in the respective body fluid or environment, or dissolute only in a delayed manner. The possibilities of control the administration of an ingredient or different ingredients may thereby by further improved.

According to a yet further preferred embodiment of the present invention, the pastes may be screen-printed in a manner such that in the resulting nutraceutical delivery system the first paste and the second paste are arranged in manner such that release of the first active nutraceutical ingredient starts before release of the second active nutraceutical ingredient with application of the nutraceutical delivery system. Printing profiles may, for example, be chosen such that the second paste is printed closer to the center or central portion of the nutraceutical delivery system. At the same time the first paste may be printed further to the edge of the nutraceutical delivery system.

Preferably, the release of the first active nutraceutical ingredient may start 1 second to 24 hours, preferably 5 seconds to 12 hours, more preferably 10 seconds to 6 hours, more preferably 20 seconds to 2 hours, more preferably 1 minute to 1 hour, and most preferred 10 minutes to 30 minutes before release of the second active nutraceutical ingredient.

Due to the particular inhomogeneous or discontinuous arrangement of the first and second pastes within the base paste, particularly concerning the dissolution direction, it may be controlled at what time the respective first and second active nutraceutical ingredients are released relative to one another. The release of the two active nutraceutical ingredients may be separated by a defined time interval depending on the spatial arrangement of the first and second active nutraceutical ingredients within the layers. Likewise, the release of the first active nutraceutical ingredient may continue when the release of the second active nutraceutical ingredient starts, depending on the spatial arrangement of the first and second active nutraceutical ingredients within the layers. Synergetic effects of the active nutraceutical ingredients may thereby be obtained. In principle, the active nutraceutical ingredients may be released to the body within seconds, minutes and/or hours. This may depend on the individual form of application.

According to a yet further preferred embodiment of the present invention, the pastes may be screen-printed in a manner such that in the resulting nutraceutical delivery system the first paste and the second paste are arranged such that a release profile of the first active nutraceutical ingredient differs from a release profile of a second active nutraceutical ingredient with application of the nutraceutical delivery system. As an example, the first active nutraceutical ingredient may be released at a rather constant rate. To the contrary, the second active nutraceutical ingredient may be released intermittently, for example. An elaborate nutraceutical delivery system may thus be designed.

According to a yet further preferred embodiment of the present invention, the total amount of the first active nutraceutical ingredient in the first paste in the resulting nutraceutical delivery system may be between 1 μg and 100 g, preferably between 10 μg and 10 g, more preferably between 100 μg and 1 g, more preferably between 500 μg and 500 mg, more preferably between 1 mg and 100 mg, more preferably between 10 mg and 50 mg. It may be comprehended that any description or explanation with regard to the first active nutraceutical ingredient may also apply to a possible second or further active nutraceutical ingredients, which may be provided in a second or further pastes of the nutraceutical delivery system.

According to a yet further preferred embodiment, one or more of the pastes may comprise a ceramic, metal, polymer, preferably a polymer acrylate, and/or any type of minerals.

According to a yet further preferred embodiment of the present invention, one or more of the pastes may comprise a disintegration agent. Such a disintegration agent may facilitate dissolution of the respective paste, that is, the cured paste.

The disintegration agent may, for example, comprise cellulose, croscarmelose sodium, crospovidone, starches, cross-linked plyvinylpyrrolidone, sodium starch glycolate, and/or sodium carboxymethylcellulose. Cellulose may preferably be micro-crystalline cellulose. Starches may preferably be modified starches.

According to a yet further preferred embodiment of the present invention, one or more of the pastes may comprise a constituent or a plurality of constituents selected from the following list: colorant, sweetener, flavor, antimicrobial preservative, chemical stabilizers which may preferably be used to increase the chemical stability of the active nutraceutical ingredient, viscosity modifiers which may be used to reduce the sedimentation of particles, cellulosic materials which may be used as viscosity enhancers in suspensions. Antimicrobial preservatives may, preferably, comprise sorbic acid, benzoic acid, parabens, scrose, and/or benzalkonium chloride. Chemical stabilizers may, for example, comprise antioxidants such as ascorbic acid or sodium metabisulfite, and/or chelators such as ethylene-diaminetetraacetic acid. Viscosity modifiers may, for example, comprise polymeric materials or inorganic materials such as clay. Cellulosic materials may, for example, comprise cellulose, cellulose ethers, and/or alginic acid.

According to a yet further preferred embodiment of the present invention, one or more of the pastes may comprise an excipient or a plurality of excipients selected from the following list: filler,, dry binder, glidant. A filler may, for example, comprise lactose, sucrose, glucose, mannitol, sorbitol, calcium carbonate, and/or cellulose. A solution binder may, for example, comprise gelatin, polyvinylpyrrolidone, cellulose derivative, and/or polyethylene glycol. A dry binder may, for example, comprise cellulose, polyethylene glycol, and/or methylcellulose. A glidant may, for example, comprise silica, magnesium stearate, and/or talc.

According to a yet further preferred embodiment of the present invention, the first paste may be screen-printed to form a geometrical shape. The shape may preferably be a tube, for example, a hollow tube, a spot, for example, a local, small cluster and/or agglomeration, an oval, such as, for example, an oval in the shape of an open circle or ellipse, a plate, and/or a polygon, for example in the shape of a square. Accordingly, the first paste may be provided in such a shape that a desired release of the first active nutraceutical ingredient may be obtained. The desired release may possibly even obtained with regard to further active nutraceutical ingredients provided in further pastes of the system. The concentration of the active nutraceutical ingredient may vary within the particular geometrical shape.

According to a yet further preferred embodiment of the present invention, the resulting nutraceutical delivery system may have the form of a tablet, a capsule, a disc, a film, an implant, a subcutaneous implant, a patch, pellets or granules. The nutraceutical delivery system produced according to the present invention may accordingly have various forms. This allows to achieve a desired administration and desired release of an active nutraceutical ingredient according to the given application.

According to a yet further preferred embodiment of the present invention, the pastes may be screen-printed such that the resulting nutraceutical delivery system may feature a structured surface. The printing profiles may, for instance, be chosen such that protrusions and/or recesses are formed within the surface of the resulting nutraceutical delivery system. The surface of the resulting nutraceutical delivery system can thereby be suitably enlarged. Eventually a high release or release rate of the respective active nutraceutical ingredient can be obtained in this manner.

The nutraceutical delivery system may, according to a further preferred embodiment, be free of any fluorescent component. The nutraceutical delivery system may also comprise a fluorescent component, for example, in order to mark different portions of the system.

It may furthermore be comprehended that the nutraceutical delivery system produced according to the present invention may not be limited to a particular active nutraceutical ingredient. In principle, any suitable active nutraceutical ingredient, which can be provided in a respective paste to be inhomogeneously arranged within a base paste, may be used in accordance with the present invention.

The active nutraceutical ingredient may, for example, be a dietary supplement, in particular a vitamin, a mineral, an herb or other botanical, an amino acid, a dietary substance for use by man to supplement the diet by increasing the total dietary intake, or a concentrate, metabolite, constituent, extract, or combination of any of the above ingredients. As an example, botanicals may be seeds, berries, leaves, roots, flowers or bark. As a further example, the active nutraceutical ingredient may be an animal extract, like a fatty acid. It may be comprehended that this list is not limiting. The nutraceutical delivery system produced in accordance with the present invention may also comprise further components or substances, for example additives or the like.

According to a yet further preferred embodiment of the present invention, the nutraceutical active ingredient may be a dietary supplement. Further preferred, the nutraceutical active ingredient may be a vitamin, a mineral, an herb or other botanical, an amino acid, a dietary substance for use by man to supplement the diet by increasing the total dietary intake, or a concentrate, metabolite, constituent, extract, or combination of any of the above ingredients.

In another preferred embodiment, the nutraceutical delivery system is free of any active pharmaceutical ingredients. In a further preferred embodiment, the nutraceutical delivery system is free of any pharmaceutical drug. In another preferred embodiment, the nutraceutical delivery system is free of any therapeutical effects.

According to a yet further embodiment of the present invention, the nutraceutical delivery system may be formed of and/or manufactured by a plurality of layers, preferably more than 2, preferably more than 3, preferably more than 5, preferably more than 10, preferably more than 15, preferably more than 20, preferably more than 25, preferably more than 50, preferably more than 75, preferably more than 100. The nutraceutical delivery system may furthermore be formed of 500 layers at the most, preferably of 400 layers at the most, 350 layers at the most, 300 layers at the most, 250 layers at the most, 150 layers at the most, 100 layers at the most, 75 layers at the most, 50 layers at the most, 25 layers at the most or 15 layers at the most.

According to a yet further embodiment of the present invention, at least one layer layers or a plurality of layers or all layers of the nutraceutical delivery system may have a constant thickness and/or a constant concentration of any ingredient or component. At least two or more layers may have identical compositions and/or dimensions.

The present invention further relates to a nutraceutical delivery system produced with a method as described herein.

According to a further aspect, the present invention relates to a system for producing a nutraceutical delivery system. The system or production system may comprise means for producing a nutraceutical delivery system in a manner as described herein. A production system for producing a nutraceutical delivery system in accordance with the present invention may comprise means for screen-printing a base paste, means for curing the base paste, means for screen-printing a first paste, said first paste being separate to the base paste and comprising an effective amount of a first active nutraceutical ingredient, and means for curing the first paste. It may again be comprehended that within the concept of the present invention, the production system may further comprise means for screen-printing a second paste, said second paste comprising an effective amount of a second active nutraceutical ingredient, as an example.

The present invention will, in the following, be described with reference to the enclosed figures. In the figures, similar features are provided with equal reference signs.

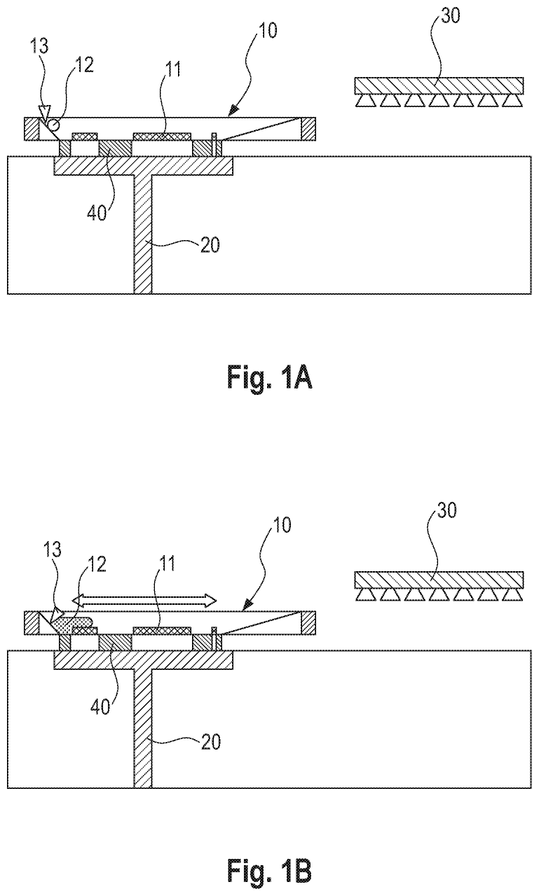

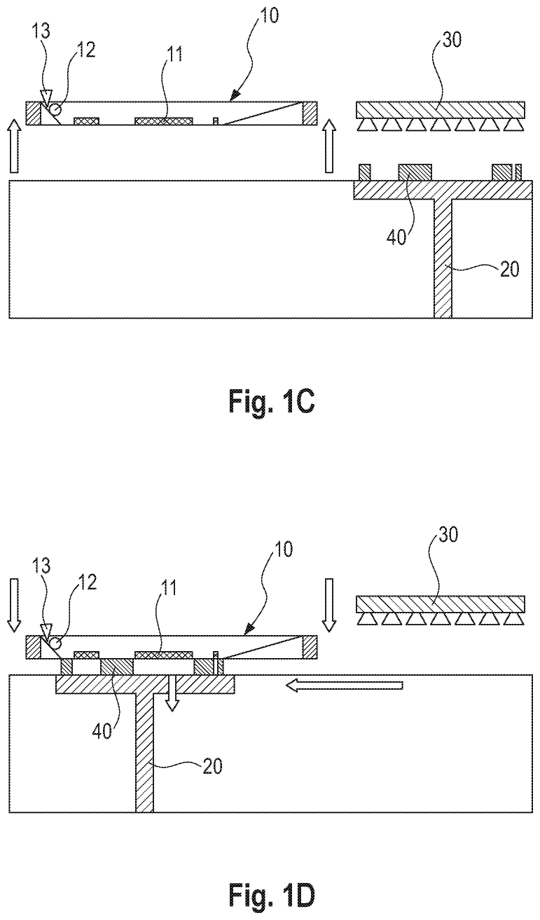

FIGS. 1A to 1D show a part of a production system for producing a nutraceutical delivery system according to an embodiment of the present invention;

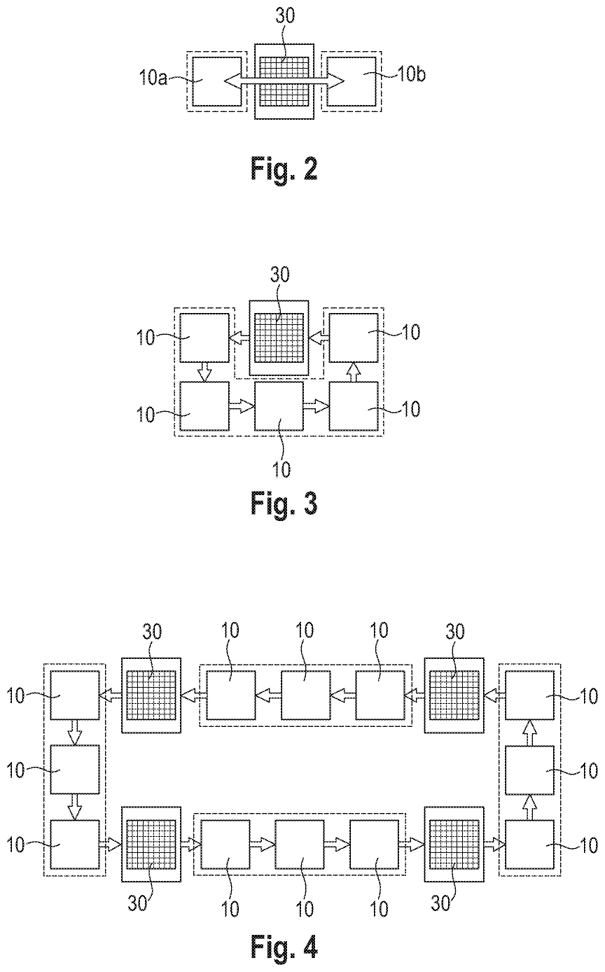

FIG. 2 shows a production system for producing a nutraceutical delivery system according to an embodiment of the present invention;

FIG. 3 shows a production system for producing a nutraceutical delivery system according to an embodiment of the present invention;

FIG. 4 shows a production system for producing a nutraceutical delivery system according to an embodiment of the present invention;

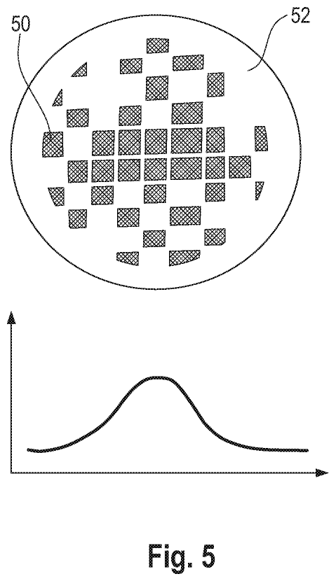

FIG. 5 shows a design of a nutraceutical delivery system produced in accordance with the present invention and the respective concentration profile;

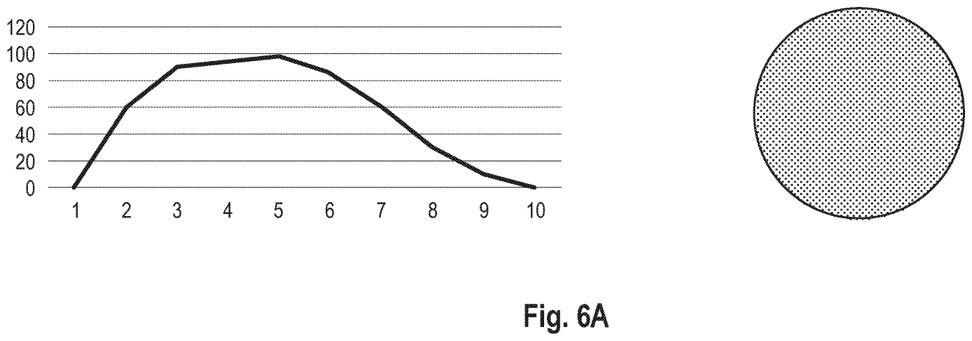

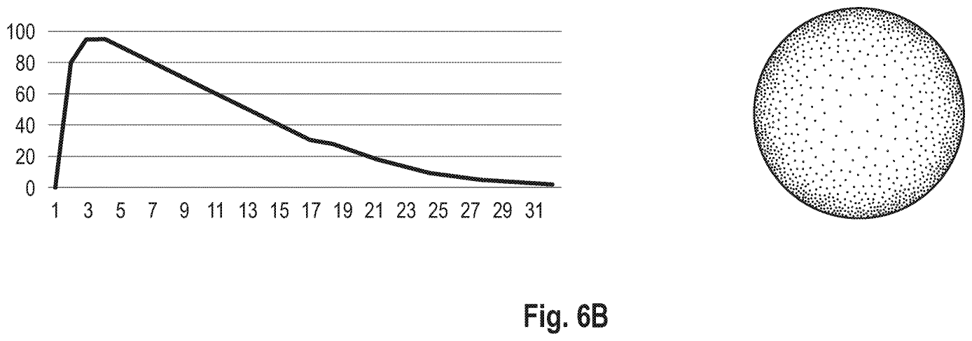

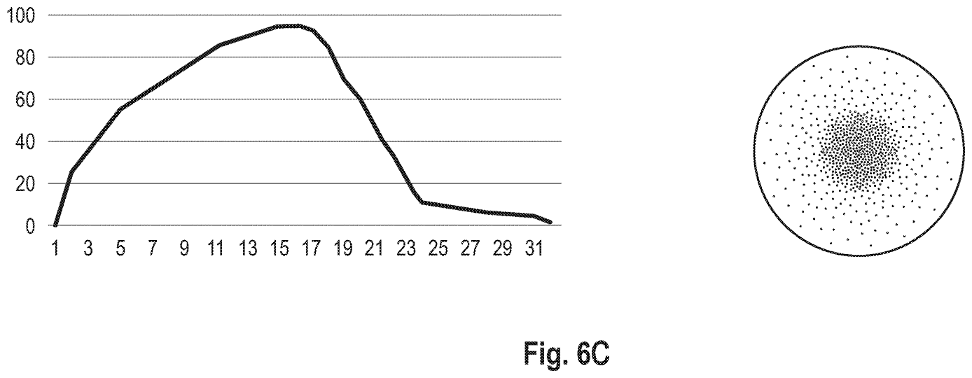

FIG. 6A shows an active nutraceutical ingredient release profiles of a nutraceutical delivery system commonly produced;

FIGS. 6B and 6C show several active nutraceutical ingredient release profiles of nutraceutical delivery systems produced in accordance with the present invention;

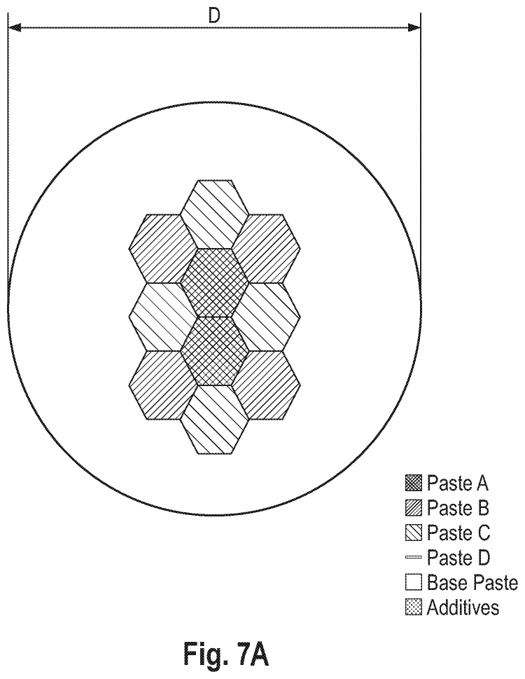

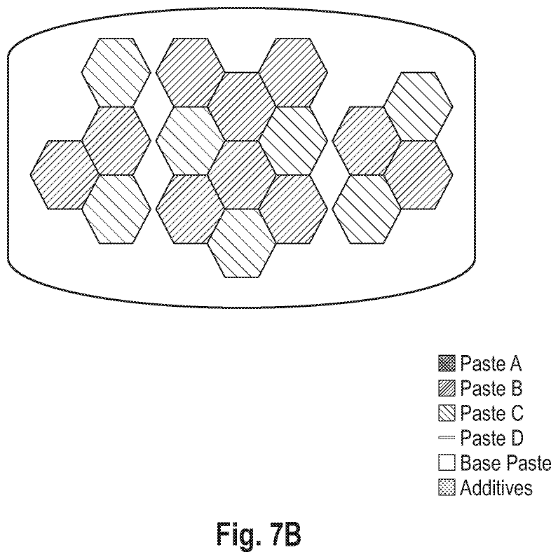

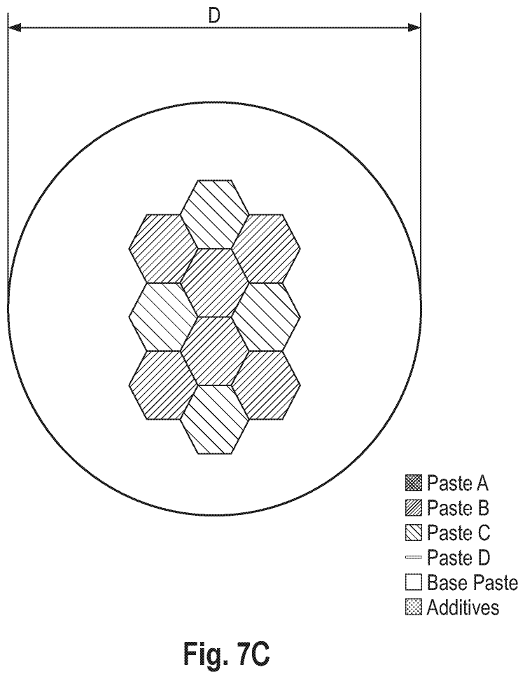

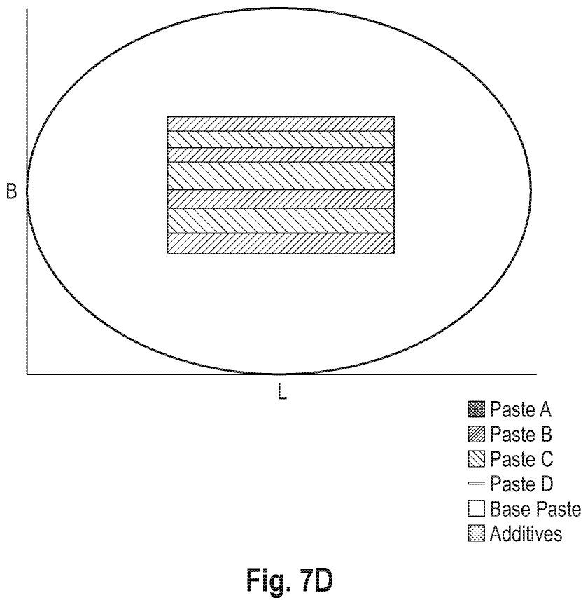

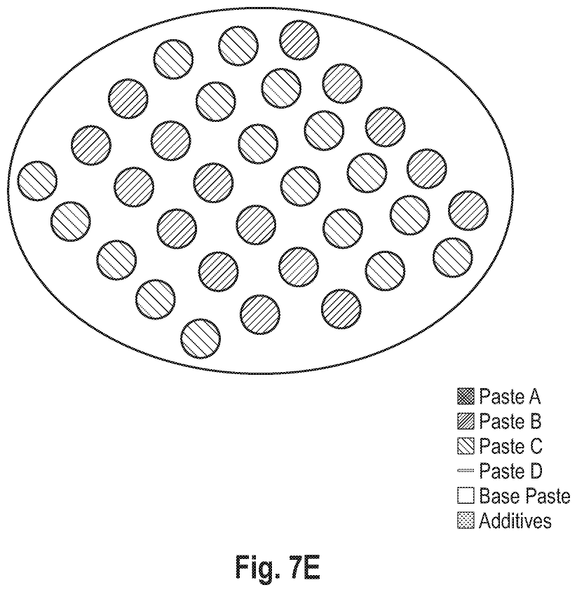

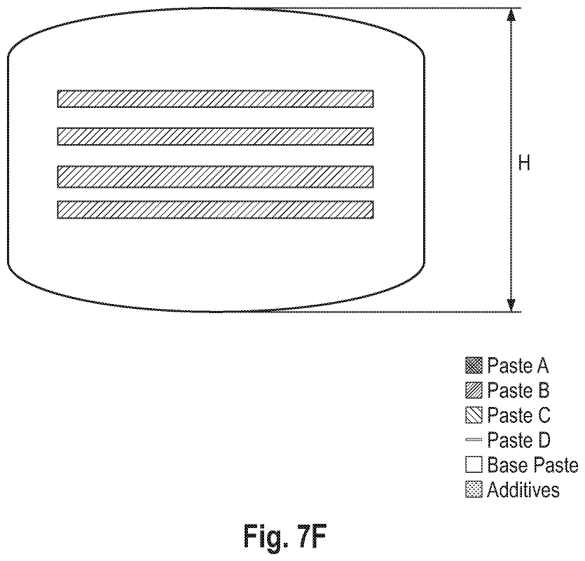

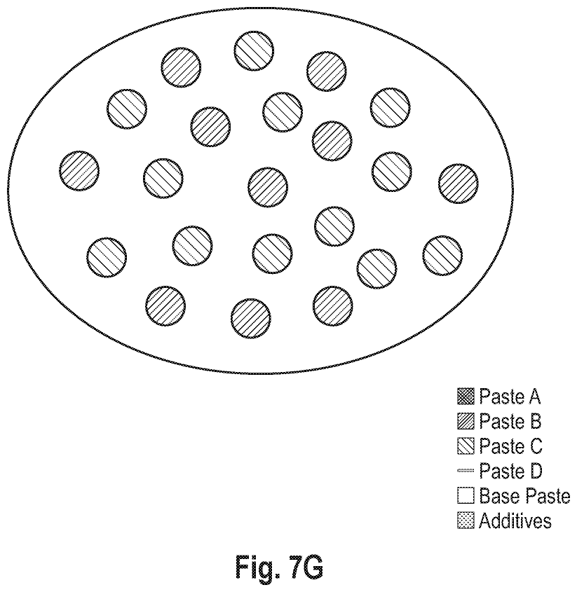

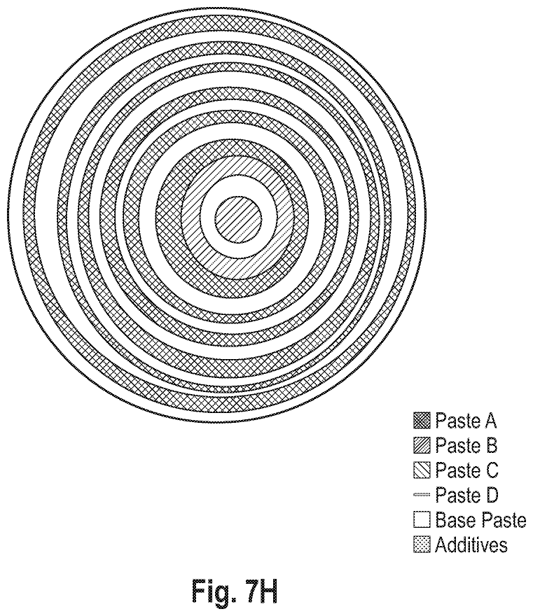

FIG. 7A to 7I show further embodiments of nutraceutical delivery systems produced in accordance with the present invention;

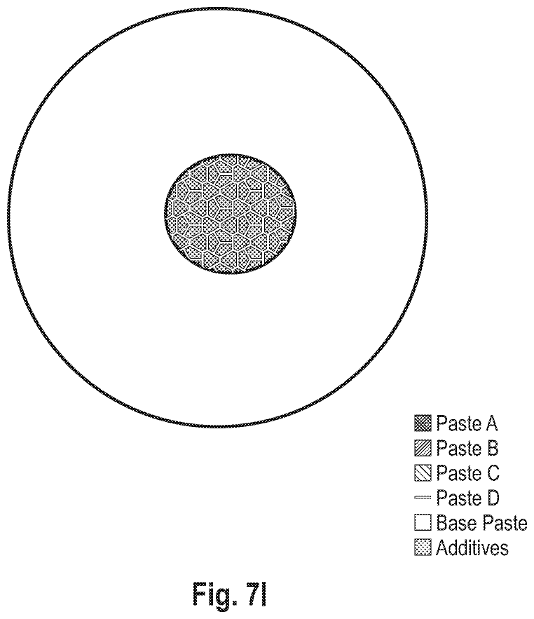

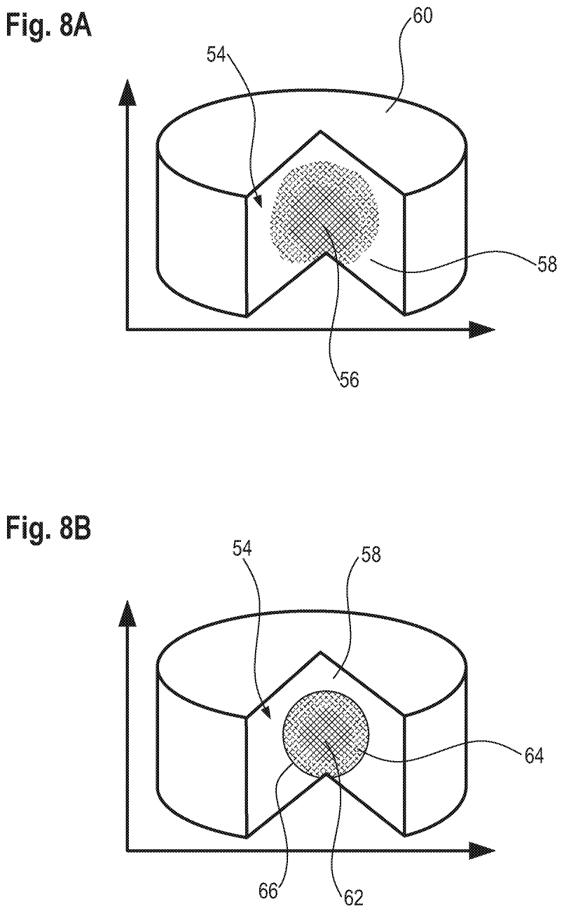

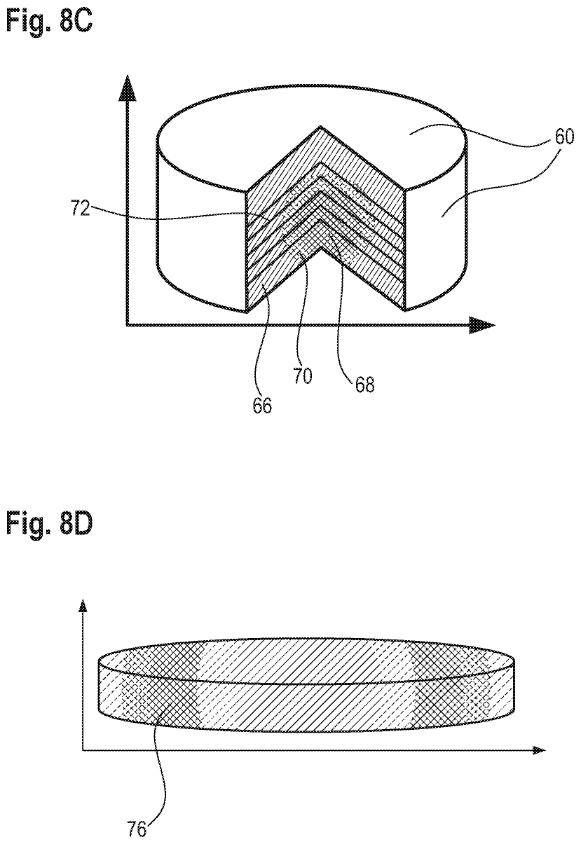

FIG. 8A to 8D show further embodiments of nutraceutical delivery systems produced in accordance with the present invention;

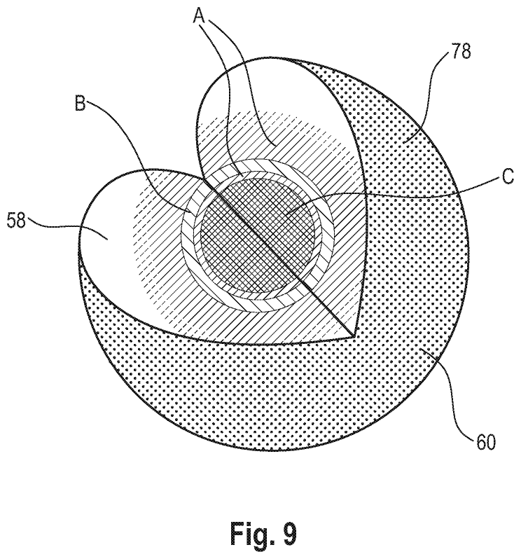

FIG. 9 shows a further embodiment of a nutraceutical delivery system produced in accordance with the present invention; and

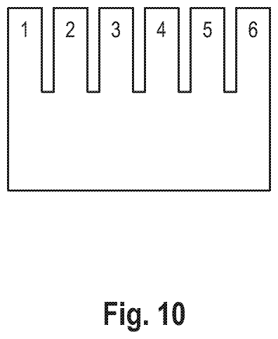

FIG. 10 shows a structured nutraceutical delivery system produced in accordance with the present invention.

FIG. 1 shows a part of a production system for producing a nutraceutical delivery system in accordance with the present invention. As may be comprehended, a screen 10 is provided, which is configured for screen-printing pastes within the meaning of the present invention. The screen 10 may, for example, be suitable for screen-printing a base paste. The screen 10 may therefore comprise a respective mask 11. The mask 11 may particular mask parts for screen-printing a desired pattern, said pattern representing a respective printing profile. The screen 10 may furthermore comprise a blade 13. The blade 13 may draw the material or paste 12 to be printed over the screen, particularly over the mask 11.

As may be further comprehended from FIG. 1A, a movable platform 20 may be provided beneath the screen 10. A built 40 is already present on platform 20. The built 40 may have been produced layer-by-layer in accordance with the present invention.

As may be comprehended from FIG. 1B, the blade 13 may draw the paste 12 along the screen 10. Thereby, a further layer of the paste 12 may be screen-printed onto the built 40. The mask 11 may mask several parts in a manner such that the paste 12 is printed only at particular locations or portions on the built 40. The arrangement of the paste 12 within the resulting nutraceutical delivery system may therefore be controlled in a precise manner.

As may furthermore be comprehended from FIG. 1C, the screen 10 may subsequently be uplifted, and the platform 20 with the built 40 comprising the additional layer of a screen-printed paste may move horizontally to place the built 40 underneath a dryer 30. The screen-printed layer may be cured by means of this dryer 30. The printed paste may thus be hardened in this manner.

The platform 20 may subsequently be moved to another screen at another printing station, in order to complete further parts of the layer by screen-printing and curing further pastes.

The platform may be returned to the shown printer and screen 10 after completion of the layer, as may be comprehended from FIG. 1D. The respective paste 12 may then again be printed on top of the built 40. The height of the platform 20 may be lowered by an amount which corresponds to the thickness of the previously build layer. Screen 10 may be moved to its lower printing position such that a further layer can be provided on top of the cured layer.

FIGS. 2-4 show different embodiments of production systems for producing a nutraceutical delivery system in accordance with the present invention. According to FIG. 2, two screen-printers 10a, 10b are provided with a dryer 30 in between. During operation of the printer 10a, a base paste according to the present invention may be printed. Said base paste may then be cured by means of the dryer 30. Subsequently a first paste may be printed by means of printer 10b, particularly at parts or potions, which are not covered by the base paste. Subsequently the first paste may likewise be cured with the dryer 30. Then the built may be moved back to the first printer 10a in order to start the production of a new layer. The three-dimensional layout of the pastes in the resulting nutraceutical delivery system can be modified, particularly by changing the meshes or printing profiles of the printers 10a, 10b, respectively.

According to the embodiment shown in FIG. 3, five printers 10 may be arranged in addition to a single dryer 30. Each of these printers may be used for screen-printing different pastes in order to eventually form a single, continuous layer. Said layer may then be cured in one step by means of the single dryer 30. A new layer may subsequently be produced on top thereof.

In accordance with the embodiment shown in FIG. 4, several printers 10 may be arranged together with several dryers 30. Thus, three successive printers 10 may print a first complete planar layer. Said layer may subsequently be cured with a respective dryer 30. Then a further planar layer may be printed on top. The latter layer may differ from the previously printed and cured layer. It may be comprehended that an according procedure may be reiterated with the further printers and dryers.

FIG. 5 shows an embodiment of a nutraceutical delivery system produced in accordance with the present invention. As may be comprehended from FIG. 5, a planar layer of the nutraceutical delivery system may extend through the nutraceutical delivery system. The paste 50 comprising the active nutraceutical ingredient and the base paste 52 may be arranged on a grid-like structure. Each “pixel” may be defined either by the active nutraceutical ingredient paste 50 or the base paste 52. As may furthermore be comprehended, the two pastes 50 and 52 may be arranged in a manner such that the density of the “active nutraceutical ingredient-pixels” may be higher at a central part or central portion of the nutraceutical delivery system. This may likewise become apparent from the active nutraceutical ingredient concentration profile also being shown in FIG. 5. The profile comprises a peak of high active nutraceutical ingredient concentration at the center of the system. The system furthermore comprises a comparatively low active nutraceutical ingredient concentration at the edges of the system. There may furthermore be a smooth transition from the low active nutraceutical ingredient concentration at the edges to the high active nutraceutical ingredient concentration at the center. Such a smooth transition does not feature any abrupt steps. The release profile of such nutraceutical delivery system with dissolution of the two pastes may be adjusted or configured in a specifically desired manner.

FIG. 6A shows a release profile of a common nutraceutical delivery system comprising a homogeneously distributed active nutraceutical ingredient. FIGS. 6B and 6C show two release profiles of nutraceutical delivery systems produced in accordance with the present invention. In FIGS. 6A to 6B, the design of the respective nutraceutical delivery system is shown next to the graphs. The nutraceutical delivery systems may be provided in a round shape. The respective nutraceutical delivery systems may be a tablet dissolving upon oral administration, for example. The graphs respectively show the release of the active nutraceutical ingredient of the respective nutraceutical delivery system over time.

As regards the graph in FIG. 6A, the design of the respective nutraceutical delivery system is in a manner such that the active nutraceutical ingredient is homogeneously distributed within and/or throughout the nutraceutical delivery system. This principle of homogeneity—homogeneity is the key feature of common prior art nutraceutical delivery systems—derives from the corresponding manufacturing processes. The respective active nutraceutical ingredient is released with dissolution of classical nutraceutical delivery systems. As a result of the dissolution characteristics of the homogeneous system and the shape of the nutraceutical delivery system, a particular and fixed release profile may be obtained. It may be comprehended from the graph in FIG. 6a that the release of the active nutraceutical ingredient increases gradually over time, then reaches a maximum, and subsequently decreases gradually.

In view of the inhomogeneous arrangement of the active nutraceutical ingredient in accordance with the present invention, different release profiles may be suitably obtained. The embodiment according to FIG. 6B is different from that in FIG. 6a, since the active nutraceutical ingredient is arranged at an edge of the nutraceutical delivery system. The principle of a homogeneous distribution of the active nutraceutical ingredient within the nutraceutical delivery system may therefore be suspended. In particular, the active nutraceutical ingredient may be inhomogeneously arranged in the nutraceutical delivery system. In the present case, a high concentration at the edge of the nutraceutical delivery system may be provided. The concentration of the active nutraceutical ingredient may smoothly decrease towards the center or central portion of the nutraceutical delivery system. With application of the nutraceutical delivery system associated with the graph FIG. 6B, the release of the active nutraceutical ingredient may be rather high or comparatively high in the beginning. Subsequently the release may decrease gradually. Such a high initial release of an active nutraceutical ingredient may be beneficial for particular applications.

In the embodiment according to FIG. 6C, the active nutraceutical ingredient may be accumulated at a central part or central portion of the nutraceutical delivery system. Accordingly, the concentration of the active nutraceutical ingredient may be comparatively high or highest at the center or a central portion of the system. The gradient of the concentration may point from the edge of the system to its center or central portion. It may be comprehended from the graph in FIG. 6C that the release increases approximately gradually over a prolonged period of time. The maximum release rate may thus be delayed in time in comparison to the common design shown in FIG. 6A. As compared to the common design, the release of the active nutraceutical ingredient may be considered to be more constant, particularly for an extended period of time. It may be comprehended that such a release profile may be beneficial for particular applications.

FIG. 7A to 7I show nine design options for nutraceutical delivery systems produced according to different embodiments of the present invention. It may be comprehended from FIGS. 7A to 7I that all these designs comprise a base paste forming the overall body of the respective nutraceutical delivery system. The base paste and/or the overall body may be considered as a matrix. Within said matrix further pastes may be arranged. These further pastes are, in FIGS. 7A to 7I, labeled as paste A, paste B, paste C and paste D. Each of said pastes may comprise an effective amount of a separate active nutraceutical ingredient. Accordingly, every one of the pastes A-D may be considered as a first paste within the meaning of the present invention. The base paste as well as the pastes A-D may be soluble in body fluids.

The design of the nutraceutical delivery system according to FIG. 7A has a round shape, as may be seen in FIG. 7A. The nutraceutical delivery system according to FIG. 7A may have the form of a tablet, a disc or the like. The system shown in FIG. 7A may have a particular diameter D . The diameter D may be, for example, 15 mm. In case of the nutraceutical delivery system according to FIG. 7A, a first paste A with a first active nutraceutical ingredient, a second paste B with a second active nutraceutical ingredient and a third paste C with a third active nutraceutical ingredient may be provided within and/or throughout the base paste. It may be comprehended that the respective active nutraceutical ingredients are not distributed homogeneously within and/or through the nutraceutical delivery system. Instead the active nutraceutical ingredients may be arranged inhomogeneously within the base paste. This is due to the fact that the pastes A, B, C are provided at particular positions within the nutraceutical delivery system. The pastes A, B, C may be provided in a polygonal shape and/or comprise a hexagonal cross section.

By applying the nutraceutical delivery system according to FIG. 7A and with dissolution thereof, the base paste may dissolves first, since the dissolution may begin at the edge of the system. After a certain period of time, paste C and then paste B may start to dissolve. Therewith, the respective active nutraceutical ingredients may be released. Subsequently, paste A may eventually start to dissolve. Therewith, the respective first active nutraceutical ingredient provided therein may be released. The different active nutraceutical ingredients may be released at different stages at different dosages after application of the nutraceutical delivery system, due to the particular arrangement of the pastes in the nutraceutical delivery system. Each active nutraceutical ingredient may be released at a particular time after applying the nutraceutical delivery system due to the particular arrangement of the different pastes within the nutraceutical delivery system according to the embodiment in FIG. 7A. A particular and individual, active nutraceutical ingredient-specific release profile may thus be achieved.

The nutraceutical delivery system according to the embodiment in FIG. 7B is formed as a tablet. The height of the tablet may, for example, amount 2.5 mm. The diameter of the tablet may amount 15 mm, for example. Two pastes B and C, each comprising an active nutraceutical ingredient, may be provided within the base paste. The pastes B and C may be provided in the base paste in an inhomogeneous manner in accordance with the present invention. By applying the nutraceutical delivery system, particular release profiles of the active nutraceutical ingredients within pastes B and C may be obtained. The release profiles may, for example, feature smooth transitions between phases of increased release.

The nutraceutical delivery system according to FIG. 7C is similar to that of the nutraceutical delivery system according to FIG. 7A. However, according to FIG. 7c the nutraceutical delivery system may comprise, only two pastes B and C be-side the base paste. The pastes B and C may each comprise an active nutraceutical ingredient. By applying the nutraceutical delivery system according to FIG. 7C, particular release profiles of the active nutraceutical ingredients contained in pastes B and C may be obtained. Said release profiles may feature smooth transitions between phases of increased release.

In the nutraceutical delivery system according to FIG. 7D, two pastes with active nutraceutical ingredients may be provided in a tube-like shape. Said pastes may likewise be provided in form of stacked plates.

The nutraceutical delivery system according to FIG. 7E has a design in which the pastes with active nutraceutical ingredients may be provided as spots within the base paste. By applying such nutraceutical delivery system, particular release profiles of the active nutraceutical ingredients contained in pastes B and C may be obtained. The release profiles may feature smooth transitions between phases of increased release.

The nutraceutical delivery system according to FIG. 7F has a design of a particular height H. Said height H may, for example, amount 25 mm. Furthermore, only one paste with an active nutraceutical ingredient may be is arranged inhomogeneously within the base paste, particularly in a tube-like manner. The paste may likewise be provided in form of plates.

The nutraceutical delivery system according to FIG. 7G is similar to the nutraceutical delivery system according to FIG. 7E. However the pastes with active nutraceutical ingredients may be arranged in a more random manner in the embodiment according to FIG. 7G. By applying the system according to FIG. 7G, particular release profiles of the active nutraceutical ingredients within pastes B and C may be obtained. Said release profiles may feature smooth transitions between phases of increased release.

The nutraceutical delivery system according to FIG. 7H may have a design, according to which the pastes with the active nutraceutical ingredients are provided or arranged in the form of circles within and/or throughout the base paste. By applying the nutraceutical delivery system, the base paste and the first paste may dissolve in an alternating manner. The dissolving in an alternating manner may occur such that the first active nutraceutical ingredient A is released intermittently, for instance, in a rather periodic manner. The second paste B may start dissolving after the first active nutraceutical ingredient is completely released Therewith the second active nutraceutical ingredient B may be released. It may be comprehended that the circles of paste A are not concentric. The circles are also not having a uniform thickness. In view of this particularly inhomogeneous arrangement, a particular release profile may be obtained. The release profile may feature smooth transitions between phases of increased release.

The nutraceutical delivery system according to FIG. 7I has a design in which a paste with an active nutraceutical ingredient is provided in a particular pattern within a matrix of additives. The latter is arranged in the base paste.

FIGS. 8A to 8D show further embodiments for nutraceutical delivery systems produced in accordance with the present invention. The overall shape of the system may be that of a round disc, in particular with a diameter of 5 -25 mm, preferably 20 mm or 15 mm. The thickness of the disc may amount to 0.5-15 mm, preferably 2 mm or 6 mm. In FIGS. 8A to 8C, a cut 54 into the tablets is provided in order to allow a view into the arrangement of the pastes within or inside the tablets.

The design of the nutraceutical delivery system according to FIG. 8A has a first paste 56 with a first active nutraceutical ingredient provided at the central part or central portion of the tablet. The central part or central portion of the tablet may be surrounded by a base paste 58. The entire tablet may be coated with a coating 60. The coating 60 may be a hydrophilic coating or be configured to have hydrophilic characteristics. The coating 60 may, for example, provide entericcoated properties. The concentration of the active nutraceutical ingredient within the tablet may be relatively high and/or highest at the center of the tablet. The concentration profile of the active nutraceutical ingredient may be such that it comprises a smooth transition, in particular a smooth transition from the edge of the tablet towards the center of the tablet. The smooth transition is indicated by different hatch typed in the FIG. 8A.

The design of nutraceutical delivery system according to FIG. 8B has a first paste 62 with a first active nutraceutical ingredient and a second paste 64 with a second active nutraceutical ingredient. The first paste 62 and the second paste 64 are provided within a base paste 58. Again, a coating 60 may be provided. The second paste 64 may be arranged in the form or a sphere. The concentration of the second active nutraceutical ingredient may be relatively high or highest on the surface 66 of the sphere. The concentration may decrease smoothly towards the center of the sphere. The first paste 62 may be provided within said sphere formed of the second paste 64. By applying the tablet and with dissolution of the pastes 62 and 64, the second active nutraceutical ingredient may be released prior to the first active nutraceutical ingredient. Both active nutraceutical ingredients may be released during a transition period.

The design of nutraceutical delivery system according to FIG. 8C may have two different active nutraceutical ingredients 66 and 68. The second active nutraceutical ingredient 68 may be provided at a central part or central portion of the tablet. The first active nutraceutical ingredient 66 may be provided around the second active nutraceutical ingredient 68. There may be an overlap of the active nutraceutical ingredients at an interface region 70 between both active nutraceutical ingredients 66 and 68. As a result, both active nutraceutical ingredients 66 and 68 may be arranged in this interface region 70. Accordingly, a smooth crossover may be achieved. The nutraceutical ingredients 66 and 68, as well as the interface region 70 are indicated by different hatch types in FIG. 8B. Further to this, layers 72 extending through the system may be provided. Said layers may be hydrophobic layers or may have hydrophobic characteristics.

The design of nutraceutical delivery system according to FIG. 8D does not have any coating or may be free of any coating. An active nutraceutical ingredient 76 may be inhomogeneously arranged within and/or throughout the tablet. As a result, areas or regions with different concentrations of the active nutraceutical ingredient may be formed or provided.

FIG. 9 shows a further design option for a nutraceutical delivery system according to an embodiment of the present invention. The system may be provided in a spherical shape. The system may, furthermore, have a hydrophobic coating 60. The coating 60 may, for example, comprise hydrophilic pores 78, preferably with sizes in the range of 1 μm to 500 μm. There may be provided a base paste 58 and three different active nutraceutical ingredients inside the nutraceutical delivery system. The active nutraceutical ingredients may be active nutraceutical ingredient A, active nutraceutical ingredient B, and active nutraceutical ingredient C. Active nutraceutical ingredient C may be provided at a central part or central portion of the nutraceutical delivery system with a peripheral pattern. Active nutraceutical ingredient C may be surrounded by the other two active nutraceutical ingredients A and B. Active nutraceutical ingredient B may thus is provided as a hollow sphere, in particular with a homogeneous distribution of the active nutraceutical ingredient. Active nutraceutical ingredient A may furthermore be inhomogeneously distributed. Active nutraceutical ingredient A may surround the active nutraceutical ingredient C. The concentration of active nutraceutical ingredient A may accordingly diminishes towards an edge of the illustrated nutraceutical delivery system. The diminishing concentration of active nutraceutical ingredient A is indicated by different hatch types in FIG. 9.

FIG. 10 shows a cross-section of a nutraceutical delivery system according to an embodiment of the present invention. As may be comprehended from FIG. 10, the surface of the nutraceutical delivery system may be structured. It is shown that six protrusions and respective recesses in between are formed on one side of the system. The surface may be increased or enlarged in this manner. The dissolution of the nutraceutical delivery system and thus the release of the active nutraceutical ingredient may be enhanced in this way. It may be comprehended that the entire surface of the nutraceutical delivery system may be structured. It is also possible that only one or several parts of the system may be structured.

It may be comprehended that the nutraceutical delivery system produced in accordance with the present invention, a particular inhomogeneous distribution of one or more active nutraceutical ingredients within and/or throughout the nutraceutical delivery system may be arranged. Thereby a desired release of an active nutraceutical ingredient or a plurality of active nutraceutical ingredients may be achieved. It may furthermore be comprehended that a prompt release or a delayed release of an active nutraceutical ingredient may be obtained. It is furthermore possible to release a particular single active nutraceutical ingredient at different dosages over a prolonged period of time. For example, a particular single active nutraceutical ingredient may be released intermittently. Thereby a release of the active nutraceutical ingredient in specific phases may be obtained.

With a single nutraceutical delivery system produced in accordance with the present invention, it is furthermore possible to obtain a release of different active nutraceutical ingredients in distinct phases. It may, for example, be possible to design the nutraceutical delivery system in a manner such that a first active nutraceutical ingredient is released prior to the release of a second active nutraceutical ingredient.

It may be comprehended that the usage of the screen-printing technique in accordance with the present invention allows for the production of such elaborate nutraceutical delivery systems with high quality, and at the same time at great quantities. The nutraceutical delivery system can therefore be produced in a mass production context.

There are numerous design options resulting from the concept of providing an inhomogeneous arrangement of one or more active nutraceutical ingredients. It may be comprehended that the above examples may be combined to obtain further elaborate designs or embodiments with release profiles optimized to the particular applications or desired effects.

1. A method for producing a nutraceutical delivery system having the form of a tablet, the method comprising the steps of:
screen-printing a base paste;
curing the base paste to form a cured base paste;
screen-printing a first paste, said first paste being separate and separately screen-printed to the cured base paste and comprising a first active nutraceutical ingredient; and
curing the first paste to form a cured first paste and thereby produce the nutraceutical delivery system,
wherein:
the base paste and first paste are screen-printed such that the cured base paste forms an overall body of the nutraceutical delivery system in the form of a matrix, and the cured first paste is arranged within said matrix formed by the cured base paste,
the base and first paste are screen-printed such that in the nutraceutical delivery system the cured first paste is inhomogeneously arranged, along three spatial directions, within said matrix formed of the cured base paste, and that in the nutraceutical delivery system the concentration of the first active nutraceutical ingredient varies throughout the nutraceutical delivery system, and
the based paste and the first paste are screen-printed such that in the nutraceutical delivery system a concentration profile of the first active nutraceutical ingredient is such that upon application of the nutraceutical delivery system, the first active nutraceutical ingredient is released at two or more dosages and release of the first active nutraceutical ingredient at one of the dosages starts 5 seconds to 12 hours before release of the first active nutraceutical ingredient at another one of the dosages.

2. (canceled)

2. The method according to claim 1, wherein the base paste and the first paste are screen-printed such that a resulting planar layer of the nutraceutical delivery system comprises both the base paste and the first paste, and the planar layer of the nutraceutical delivery system is produced by the steps of:
screen-printing and curing the base paste to partially form the planar layer, and
screen-printing and curing the first paste separate to the base paste to form a remainder of the planar layer.

4. (canceled)

5. (canceled)

6. The method according to claim 1, wherein the base paste is screen-printed by a screen-printer, and the first paste is screen-printed using a separate screen-printer, and/or the base paste and the first paste are cured with a shared curing device.

7. (canceled)

8. (canceled)

9. (canceled)

10. (canceled)

11. (canceled)

12. The method according to claim 1, wherein the base paste and the first paste are screen-printed such that in the nutraceutical delivery system a concentration of the first active nutraceutical ingredient is relatively high and/or highest at a center of the nutraceutical delivery system, at an edge of the nutraceutical delivery system, or at an intermediate region of the nutraceutical delivery system.

13. The method according to claim 11, wherein the base paste and the first paste are screen-printed such that in the nutraceutical delivery system the concentration profile of the first active nutraceutical ingredient is a gradient of concentration that increases towards or increases away from a center of the nutraceutical delivery system.

14. The method according to claim 1, wherein the base paste and the first paste are screen-printed such that in the nutraceutical delivery system the concentration profile of the first active nutraceutical ingredient within and/or throughout the nutraceutical delivery system comprises a smooth transition to an area and/or portion of increased concentration.

15. The method according to claim 1, wherein the base paste and the first paste are screen-printed such that in the nutraceutical delivery system the concentration profile of the first active nutraceutical ingredient within and/or throughout the nutraceutical delivery system comprises more than one area and/or portion of increased concentration.

16. The method according to claim 1, wherein the base paste and the first paste are screen-printed such that in the nutraceutical delivery system a variation of concentration of the first active nutraceutical ingredient within and/or throughout the nutraceutical delivery system is at least 5%.

17. The method according claim 1, wherein the base paste and the first paste are screen-printed such that in the nutraceutical delivery system a variation of concentration of the first active nutraceutical ingredient within and/or throughout the nutraceutical delivery system is at most approximately 100%.

18. The method according to claim 1, wherein the base paste and the first paste are screen-printed such that in the nutraceutical delivery system the concentration profile of the first active nutraceutical ingredient is such that upon application of the nutraceutical delivery system, the first active nutraceutical ingredient is released from the nutraceutical delivery system at a predetermined release characteristic and/or release profile, which comprises a section with a release at a constant rate.

19. The method according to claim 1, wherein the first paste and the base paste are screen-printed such that in the nutraceutical delivery system the concentration profile of the first active nutraceutical ingredient is such that release of the first active nutraceutical ingredient at one of the dosages starts 10 seconds to 6 hours before release of the first active nutraceutical ingredient at another one of the dosages.

20. The method according to claim 1, wherein the base paste and the first paste are screen-printed such that the base paste envelops and/or encloses the nutraceutical delivery system and/or in that the first paste is not arranged at an outer face of the nutraceutical delivery system and/or in that the outer face of the nutraceutical delivery system is free of the first paste.

21. The method according to claim 1, the method further comprising the steps of:
screen-printing a second paste, said second paste being separate from the base paste and the first paste and comprising a second active nutraceutical ingredient; and curing the second paste.

22. The method according to claim 21, wherein the based paste, the first paste, and the second paste are screen-printed such that a resulting planar layer of the nutraceutical delivery system comprises the base paste and the first paste and the second paste.

23. The method according to claim 22, wherein the planar layer of the nutraceutical delivery system is produced by the steps of:
screen-printing and curing the base paste to partially form the planar layer,
screen-printing and curing the first paste being separate to the base paste to partially form the planar layer,
screen-printing and curing the second paste being separate to the base paste and the first paste to partially form the planar layer.

24. (canceled)

25. The method according to claim 21, wherein the base paste, the first paste, and the second paste are screen-printed such that in the nutraceutical delivery system the second paste is inhomogeneously arranged in and/or throughout the base paste.

26. The method according to claim 21, wherein the base paste, the first paste, and the second paste are screen-printed such that in the nutraceutical delivery system the concentration profile of the first active nutraceutical ingredient throughout and/or within the nutraceutical delivery system is different than a concentration profile of the second active nutraceutical ingredient throughout and/or within the nutraceutical delivery system.

27. The method according to claim 21, wherein the base paste, the first paste, and the second paste are screen-printed such that upon application of the nutraceutical delivery system, release of the first active nutraceutical ingredient starts before release of the second active nutraceutical ingredient, optionally wherein the release of the first active nutraceutical ingredient starts 5 seconds to 12 hours before release of the second active nutraceutical ingredient.

28. The method according to claim 21, wherein the base paste, the first paste, and the second paste are screen-printed such that upon application of the nutraceutical delivery system, a release profile of the first active nutraceutical ingredient is different from a release profile of the second active nutraceutical ingredient.

29. (canceled)

30. (canceled)

31. The method according to claim 1, wherein the first active nutraceutical ingredient comprises one or more of vitamins, minerals, herbs or other botanicals, amino acids, or dietary substances for use by a human to supplement the diet by increasing the total dietary intake.

32. (canceled)

33. (canceled)

34. (canceled)
